(19) United States
(12) Patent Application Publication
(10) Pub. No.: US 2014/0114115 A1
(43) Pub. Date: Apr. 24, 2014

(54) GAMMA SOURCE TRACKING SYSTEM

(71) Applicant: Nucletron Operations B.V., Veenendaal (NL)

(72) Inventor: Paulus Cornelis Hendrikus Maria Krechting, Veenendaal (NL)

(73) Assignee: Nucletron Operations B.V., Veenendaal (NL)

(21) Appl. No.: 14/060,818

(22) Filed: Oct. 23, 2013

Related U.S. Application Data
(60) Provisional application No. 61/717,896, filed on Oct. 24, 2012.
(60) Provisional application No. 61/783,943, filed on Mar. 14, 2013.

(30) Foreign Application Priority Data
Oct. 24, 2012 (NL) 2009686

(51) Int. Cl. A61N 5/10

(52) U.S. Cl. CPC A61N 5/1031; A61N 5/1001
USPC 600/3; 600/1; 378/207

(57) ABSTRACT

Embodiments of the disclosure relate to a method for reconstructing a spatial position of a conduit arranged to accommodate a radiotherapeutic radioactive source. The method includes displacing an object emitting radiation inside the conduit prior to the administration of a treatment, detecting said radiation using detectors; generating data upon detecting the said radiation using a processor; and reconstructing the spatial position of the conduit by the processor based on the said data to identify a delivery path of the treatment.

### PRIORITY

This patent application claims the benefit of priority under 35 U.S.C. §190 to U.S. Provisional Application No. 61/717,896, filed on Oct. 24, 2012, and U.S. Provisional Application No. 61/783,943, filed on Mar. 14, 2013, each of which is incorporated herein by reference in its entirety. This patent application also claims the benefit of priority under 35 U.S.C. §119 to The Netherlands Patent Application No. 2009686 filed on Oct. 24, 2012, which is incorporated herein by reference in its entirety.

### FIELD OF THE INVENTION

The invention relates to a method for reconstructing a spatial position of a conduit arranged to accommodate a radiotherapeutic radioactive source.

The invention further relates to an apparatus for enabling quality assurance of a brachytherapy treatment.

The invention still further relates to an afterloader device.

### BACKGROUND OF THE INVENTION

In clinical practice brachytherapy applications are gaining importance. In the course of a brachytherapy treatment a radioactive source, usually a gamma emitter, is introduced into a target volume of a patient by means of a suitable conduit, such as a brachytherapy applicator, an interstitial needle, a catheter, or the like. The radioactive source may be introduced manually or using an afterloader device. Generally, the afterloader device is used for providing the radioactive source or sources inside the patient for a given (short) period of time inside suitable pre-positioned conduits. In such a case, the gamma source may be a high dose rate source or a low dose rate source. Alternatively, the sources (seeds) may be provided inside the target volume of the patient for a prolonged time (several hours) or even for permanent dwelling (until the full decay). Such sources may be low dose rate sources.

It is a disadvantage of the contemporary brachytherapy technique that the actual source position inside the volume is verified indirectly. In particular, it is a disadvantage of the contemporary brachytherapy technique that no specific information is present about the spatial position of the conduits and/or about a deformation of the conduit after it has been inserted into a human body.

However, it will be appreciated that interstitial needles, applicators or other suitable conduits arranged to be positioned within a patient's tissue may be introduced under application of a substantial force. As a result such conduits may accidentally deform, which would inevitably alter the trajectory of the radioactive source inside the patient in comparison with the pre-planned trajectory, calculated for non-deformed conduits.

### SUMMARY OF THE INVENTION

It is an object of the invention to provide a method of reconstructing actual spatial position of the conduits for brachytherapy in real time accurately. It is a further object of the invention to provide a method of recalculating a dose delivery plan in real time for established actual positions of the conduits and the sources.

To this end method for reconstructing a spatial position of a conduit arranged to accommodate a radiotherapeutic radioactive source, the method comprising:
displacing an object emitting radiation inside the conduit,
detecting said radiation;
generating data upon detecting the said radiation;
reconstructing the spatial position of the conduit based on the said data.

It is found that the actual geometry of the inserted conduit may be accurately reconstructed in real time when a suitable object emitting radiation is displaced within the conduit and the radiation emanating from the object is detected. It will he appreciated that for resolving the 3D position of the radiation emitting object (and, thus, the conduit) at least two detectors registering radiation from the object in real time are required. Preferably, for the detectors suitable position sensitive devices (PSD). The detectors may be arranged to detect the radiation emanating from the object continuously, or for suitable pre-determined intervals.

In an embodiment of the method, the object generates electromagnetic radiation, such as gamma rays or, alternatively, it may generate ultrasonic rays. In case when the object is generating gamma rays the PSD may cooperate with a suitable scintillator crystal which is arranged to emit light upon interception of the incoming gamma ray. Preferably, the detectors are collimated in order to reduce scattering component. In case when the object is emitting ultrasonic waves the suitable detector is arranged to receive incoming sound.

In a further embodiment of the method according to the invention the conduit is an interstitial needle, an applicator, or a catheter the method further comprises the steps of:
accessing a pre-plan calculated for the conduit;
calculating a net dose distribution based on the pre-plan and the reconstructed spatial position of the conduit.

For example, it is possible to use the actual source for determining the spatial position of the conduits. However, it will be appreciated that it may be advantageous to use a mimic source, which may represent geometry of the actual source, but which has a substantially lower activity than the radiotherapeutic source.

In both cases, it is possible to determine the actual position of the conduit, and based on that, to determine the actual therapeutic dose distribution. It will be appreciated that when such dose distribution is prospectively determined, it is possible to modify the dose plan, for example, in terms of source dwell positions, in order to compensate for the occurred unforeseen displacement of the conduit or unforeseen deformation of the conduit.

For example, in accordance with the present embodiment, first, the spatial position of the conduit is determined using the displaceable object. It may be preferable to use a continuous acquisition of radiation emitted by the object in order to acquire position information about the conduit along its full lengths. Alternatively, especially for the situation, when the actual radiotherapuetic plan is carried-out and a number of the pre-planned dwell positions of the radiotherapeutic source are determined, data acquisition from the object may be carried out at the pre-planned dwell positions of the actual radiotherapeutic source. As a result, accurate data are provided for checking the expected dwell positions of the actual radiotherapeutic source and the net dose distribution may be calculated for the thus expected dwell positions. Preferably, the conduit is visualized as a three-dimensional map, which may increase the transparency of the procedure to a medical specialist. It will be appreciate, however, that it is also possible that the three-dimensional map is stored digitally in a suitable computer.

In a further embodiment of the method according, to an aspect of the invention, said map is obtained using a measurement cable of an afterloading device.

It is found to be particularly advantageous to use the measurement cable of the afterloading device for purposes of determining the three-dimensional position of the conduit. This technical feature is based on the insight that it is advantageous to use the available motorized cable provided in the afterloading device for transporting the object inside the conduit.

It will be further appreciated that for a radiotherapy treatment a number of conduits may be used, for example a number of catheters, applicators or conduits. Accordingly, the procedure of establishing the actual position of the conduit in three-dimensions may be repeated for each such conduit.

More preferably, the displaceable object used for determining the actual position of the conduit may be arranged to mimic the actual radiotherapeutic source. Such mimicking may be effectuated in terms of geometry and/or in terms of emitted radiation. For example, the object may comprise the same radioisotope as is used in the actual radiotherpeutic source, yet having a considerably less activity. For example, the level of radioactivity object may be 10 to 100 times weaker than the level of the actual radiotherapeutic source.

In a still further embodiment of the method according to a further aspect of the invention, in which a plurality of conduits is used, wherein each conduit is assigned with a pre-determined three-dimensional position according to the treatment plan, the method further comprises the step of verifying whether the actual three-dimensional map of each conduit matches the pre-determined three-dimensional position.

This feature is particularly important for determining whether the conduits are placed correctly and whether they are not interchanged by chance. Accordingly, when the map of all of the conduits is provided, it is possible to visualize, or to check otherwise, whether both the position and the prescription number of the conduits match the pre-plan. These factors are particularly important for maintaining consistency of the radiotherapeutic plan, as the radioactive source may have different dwell positions and/or different dwell times inside each conduit. Therefore, interchanging of the conduits, such as needles, may cause dramatic perturbation in the delivered dose.

According to a still further embodiment of method it further comprises the steps of:
displacing the radiotherapeutic radioactive source inside the conduit,
detecting radiation emitted by the radioactive source;
reconstructing the total dose delivered by the radiotherapeutic radioactive source.

It is found that the detectors which are suitable for detecting radiation from the displaceable object emitting gamma rays may be used for detecting radiation emitted by the actual radiotherapeutic source during treatment. As a result, an efficient and accurate registration method of the delivered dose is provided, which may be used for improve quality assurance of the brachytherapy treatment.

In the apparatus according to an aspect of the invention comprises:
a displaceable object adapted to be displaced inside the conduit, said object emitting radiation,
a detector capable of detecting radiation from the object;
a processor for reconstructing the spatial position of the conduit based on the output of the said detector.

Preferably, the object is arranged to mimic a radiotherapeutic radioactive source used for treatment, at least in terms of geometry. It is preferable to use for the procedure of determination of the conduit dimensions to use a source which has a substantially lower dose rate than the actual radiotherapeutic radioactive source. For example, such mimic source may be provided on a dummy guidewire of an afterloading apparatus which is usually send out for checking accessibility of the conduits for receiving the actual radioactive source. More preferably, the processor of the apparatus according to a further aspect of the invention is adapted for reconstructing the total dose delivered by the radiotherapeutic radioactive source accommodated and displaced in the conduit. Those skilled in the art would readily appreciate that the determined spatial position of the conduit can readily be used in a dose planning system for re-calculating the dose plan based on the adapted source dwell positions. It will be appreciated that a possible deformation of the conduit may cause a substantial change in the convoluted dose delivery pattern, especially when a plurality of)thus deformed) conduits is used.

The afterloader device for effectuating a brachytherapy treatment using a radioactive source, comprised the apparatus as is described with reference to the foregoing.

These and other aspects of the invention will be discussed with reference to Figures, wherein like reference numbers refer to like elements. It will be appreciated that the figures are provided for illustrative purposes only and may not be used for limiting the scope of the appended claims.

### BRIEF DESCRIPTION OF THE DRAWINGS

FIG. 1 presents in a schematic way an embodiment of an apparatus for enabling quality assurance of a brachytherapy treatment according to an aspect of the invention.

FIG. 2 presents in a schematic way a further view of the embodiment of the apparatus of FIG. 1.

FIG. 3 presents in a schematic way an embodiment of hardware system architecture according to an aspect of the invention.

FIG. 4 presents in a schematic way an embodiment of a displaceable object according to an aspect of the invention.

### DETAILED DESCRIPTION OF THE DRAWINGS

A gamma source tracking system according to the exemplary disclosure may work in conjunction with an afterloader to provide three-dimensional (3D) imaging of brachytherapy treatment implants inserted into a patient's body. The treatment implant may consist of one or more conduits configured to be connected to an afterloader and to deliver treatment, in the form of a radioactive source, from the afterloader to a treatment area, e.g., a tumour. A gamma source tracking system may he used to provide a technician with an accurate image of the location, positioning, and/or integrity of the conduits implanted within the body. Such an image may be useful prior to the administration of treatment to promote delivery of the treatment to the correct location to ensure that the treatment will be able to successfully pass through the conduits. This image may also be stored for future use as a reference guide. This may allow a technician to verify that consistent placement, and therefore treatment location and dosage, is achieved over multiple treatment sessions. If an operator accidently switched the location of one or more conduits, then automatic or manual comparison of the new image with the reference image would highlight the inconsistency, allowing for correction prior to treatment administration. Additionally, such imaging may he used to check that the correct dose is in fact being administered to the correct location, thus serving as a treatment verification tool, and to check the integrity of the conduits' connections with the afterloader. In at least these ways, a gamma source tracking system may act as a quality assurance means during brachytherapy treatment.

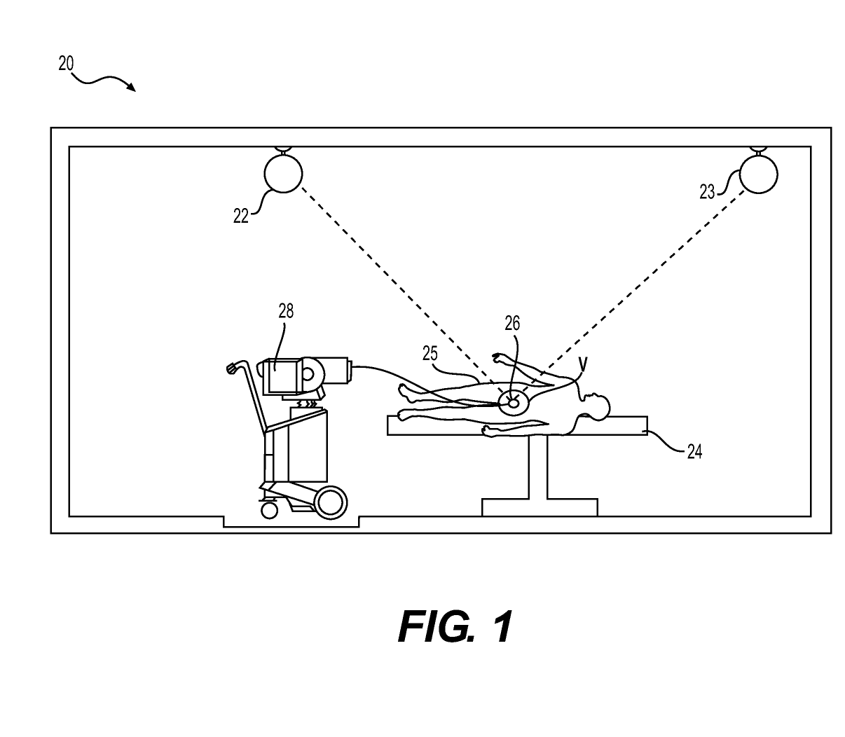

FIG. 1 presents an exemplary embodiment of an apparatus for enabling quality assurance of a brachytherapy treatment. In this particular embodiment, the apparatus comprises a tracking system using array-detectors. It will he appreciated that the tracking system may be adapted to use the drive mechanism of an afterloader to displace a radioactive source, an electromagnetic source, or an ultrasonic source, to track the displacement of the object as it passes through each conduit of an implant within a patient. Exemplary suitable afterloaders are described in commonly owned U.S. Pat. Nos. 7,645,224 and 8,273,007, both incorporated herein by reference. The displaced source will herein be referred to as an ‘object.’ Those skilled in the art would readily appreciate which detectors are suitable for detecting each type of the radiation, and thus for tracking each type of source displaced.

In one embodiment, the apparatus for enabling quality assurance of a brachytherapy treatment may be built into an afterloader device used for effectuating brachytherapy. Accordingly, the tracking system object may he displaced using the drive mechanism of the afterloader via attachment to a drive cable, which is mounted on the afterloader drive unit. By electromechanically rotating the drive unit, the object may be moved through the conduits of a brachytherapy applicator. It will be appreciated, however, that, alternatively, the apparatus may be a stand alone system, having a controllable drive for displacing the object emitting radiation inside a suitable conduit.

In accordance with an aspect of the disclosure, a patient 25 is suitably positioned inside a treatment room 20. The patient 25 is usually suitably positioned on a table 24 for receiving a radioactive gamma source (not shown) from an afterloading device 28 for local treatment. One or more conduits, or an applicator containing one or more conduits, implanted within patient 25 may be configured to receive treatment from afterloader 28 and deliver the treatment to a predetermined treatment area within patient 25.

Prior to treatment, a suitable object 26 may be connected to a drive mechanism of an afterloader 28 via a drive cable and aligned with a conduit of a brachytherapy applicator or implant. Using the drive mechanism, the object may be displaced along the length of the conduit. In accordance with an aspect of the disclosure, an apparatus may be used to determine the 3D position of object 26 as it is displaced along the conduit. Such an apparatus may include, for example, pinhole detectors 22, 23 configured to detect displaceable object 26. The displaceable object 26 may then be moved along the pre-positioned conduits (not shown), and the detectors 22, 23 detect radiation emanating from the object. Data from the detectors may be supplied to a suitable processor (not shown) for calculating the position of the object and, thus, the conduit, in 3D. Any suitable number of detectors may he used. To achieve 3D imaging, 2 or more detectors may be used to calculate the 3D location of object 26. If more than 2 detectors are used, the detectors may continue to track the 3D location of object 26 even if object 26 becomes obscured from one of the detectors or malfunctions.

It will be appreciated that the detectors 22, 23 may be mounted on a ceiling of the room 20. However, any suitable mounting position may be used. For example, the detectors 22, 23 may be mounted on the table 24, which may be advantageous as the flux from the object 26 decreases inversely proportional to a square of a distance to the source. In some embodiments, detectors 22, 23 may be mounted approximately 1 to 2 meters from the object 26 that they are configured to detect. Those skilled in the art will readily appreciate an optimal distance between the volume V in which the object is transported and the object 26 which may he determined based on a given source activity.

In one embodiment, a calibration of the detectors 22, 23 may be done prior to use. For example, a reference point in the treatment room 20 may be selected for defining an origin of a coordinate system. Such reference point may be referred to as the isocentre. The detectors 22, 23 may be mounted in the room in such a way that they cover a cylindrical volume around the isocentre. The x, y, and z coordinates of the isocenter may be (0, 0, 0). This may be the location of the object 26 at a given starting position. The detectors may then check the distance and position by placing the object 26 in fixed positions along the x, y, and z directions, so that the direction and distance may be calibrated in 3D. Any suitable calibration technique may be used.

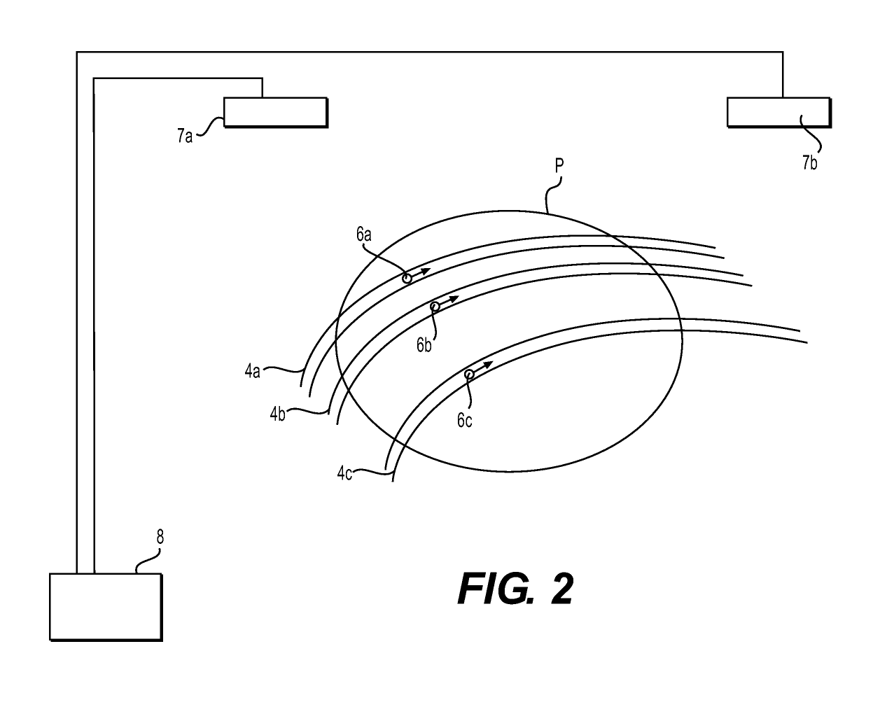

FIG. 2 presents in a schematic way a further view of the embodiment of the apparatus of FIG. 1. In this example embodiment, three conduits 4a, 4b, 4c are pre-inserted into a patient P. In order to effectuate brachytherapy, a pre-plan may be calculated that defines the desirable positions of the conduits 4a, 4b, 4c for treatment of a target area within the patient P, and that also defines the corresponding desirable dwell positions and dwell times of the radioactive source.

Once the detectors 22, 23 are calibrated to verify the actual spatial positions of the conduits 4a, 4b, 4c, a displaceable emitting object may he provided. The displaceable object may be sized to he transported inside the respective conduits (6a, 6b, 6c). The displaceable object may be connected to the drive mechanism of afterloader 28 via a drive cable, and the object may be displaced along each of conduits 4a, 4b, 4c one at a time. The object may he displaced down the first conduit, and its displacement from the isocentre position may be measured as it passes through the conduit. The object may then be retracted from that conduit and moved to the next conduit, where the process may be repeated. During this transport, radiation emitted by the object may be detected by the suitable detectors 7a, 7b. Signals detected by the detectors may be further processed by a suitable processor 8. The processor 8 may form part of a dose planning system based on the received data from the detectors 7a, 7b and may be used to calculate the respective positions of the conduits 4a, 4b, 4c in space based on the signals.

In one embodiment, the processor 8 is arranged to create a three-dimensional map of the conduits for verifying their position. The position may be compared to an index, for example, a similar reference image from a prior treatment session or a intended treatment map, to verify a correspondence between the conduit's index (such as a number in the pre-plan) and its actual position. By measuring the positions of the source object in the conduits, the correct position of each conduit may be tested, the connection between the afterloader and the conduit may be checked, and the accuracy of the remote afterloader positioning may be tested.

In some embodiments, if improper positioning is detected, treatment may be postponed until the conduits may he repositioned. In some embodiments, an improper connection with the afterloader 28 may be detected and the connection may then be adjusted. In some embodiments, the tracking system may be used in real-time as the conduits are inserted into the patient in order to detect correct placement and make adjustments in real time.

In some embodiments, the obtained 3D map of the actual positions of the conduits may be further used by a suitable planning system to calculate the resulting dose after taking into account the actual position of the conduits. The whole process may be suitably automated, which may lead to an overall reduction of the patient handling time, more accurate dosing regimens, and improved patient treatment.

In addition, because the process may be fully automated and quantitative, errors in connection between the afterloader device and the conduits (e.g., using an incorrect transit tube) may be eliminated, as the correspondence between the index of the conduit and its actual position is often known. This may be achieved by use of a reference image, as discussed above. Prior to the first treatment, a full 3D map may be created using an object and the gamma source tracking system of the positioning of the implant and conduits, and this map may be stored as a reference. Such a 3D map may be taken at the beginning of subsequent treatment sessions, and the newly obtained map may be compared to the previous reference maps. This would allow a technician to detect, e.g., if two conduits were switched or the conduits were misplaced, prior to the administration of radioactive treatment.

Next, an embodiment according to the present disclosure may be further used for recording the actual dose delivered during the treatment and its location, as the detectors may also be suitable for detecting gamma radiation emitted by the actual radiotherapeutic source used for treatment. The apparatus may be capable of tracing the displacement of the radiotherapeutic source inside the conduits. This data may be input into the planning system for re-calculating the actually deposited dose based on the actual trajectory of the radiotherapeutic source. These actions may be carried out in real time, which may provide a clinician with the opportunity to interrupt the treatment should a substantial discrepancy, for example, larger than 5%, be detected between the prescribed dose distribution and the actual dose distribution. In some embodiments, the trajectory of the source may he detected, and if the actual trajectory deviates by a pre-determined value from the pre-planned trajectory, the treatment may be interrupted.

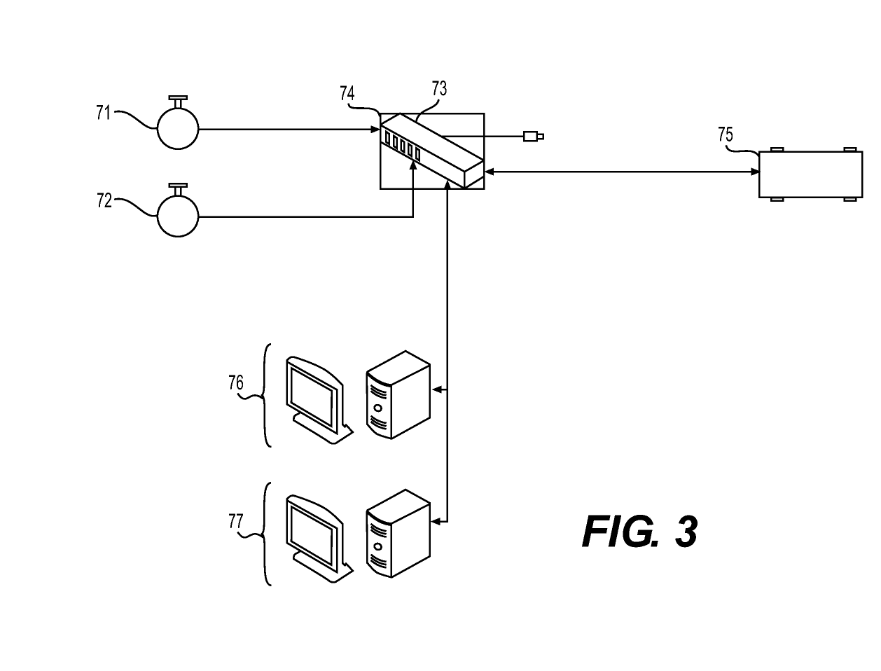

FIG. 3 depicts an exemplary embodiment of a suitable hardware system architecture for enabling determination of the spatial position of a conduit in real time. It will be appreciated that there are numerous options for implementing the system according to the present disclosure. In one embodiment, the output of a position sensitive detector (PSD), such as detectors 22, 23 shown in FIG. 1, is connected to a field programmable gate array (FPGA) via a suitable A/D converter. It will be appreciated that in this embodiment, each PSD may communicate with its own FPGA. The output of the FPGA is connected to a PC. The FPGA may be arranged to only calculate the x & y coordinates of the PSD itself. The position of the PSDs in relation to each other may then be used to determine the 3D coordinate(s). The PSDs may include a scintillator configured to transform radiation into physical light. A light emitting diode (LED) may be incorporated in this system and used for calibration purposes to emit light and mimic the radiation source. The PC or an FPGA may then be used to calibrate and calculate the position of the LED in the work area.

In the embodiment of FIG. 3, two PSDs 71, 72 may be placed at the ceiling of the treatment room. A PoE (Power over Ethernet) switch 74 may be placed in the ceiling and connected to a PC 73. The two PSD are connected to the PoE switch via Ethernet. In other embodiments, the components may be connected wirelessly and no switch 74 may be needed.

This embodiment may have the following advantages:
The calculation of the x, y & z coordinates is performed on one central location in the system.
The system is modular. Any suitable number of detectors can be installed; the limit is the number of IP addresses the DHCP server on the PC is configured to handle.

The calculation of the 3D coordinates may be fast, given the PC's calculation power and software libraries.
No data may be stored offline in the FPGA.
Fast connection between the FPGAs and the PC through a standardized 100 Mbit/s Ethernet interface.
The detectors may be powered through PoE. This standard is EMC and ESD certified.

In another embodiment, the system uses one FPGA to read data from two detectors. The detectors may be connected with the FPGA through a serial peripheral interface (SPI) bus. The FPGA may be calibrated to calculate the distance from the isocenter and then send the x, y and z coordinates to the PC. The PC may then show the calculated coordinates.

This embodiment may have the following advantages:
A single FPGA may be used for the entire system which may reduce the cost per sensor.
The full calculation power of the FPGA may be used.

It will be appreciated that the choice between the first and the second embodiments may depend on the demands of a particular situation. In some embodiments, the system may further comprise an embedded PC 75, which may function as a position server. The output of the FPGA 73 may be provided to a planning system 76 for calculating the actual dose distribution inside the patient based on the real time positions of the gamma source determined using the PSD's 71 and 72. Secondly, the output of the FPGA 73 may he provided to the afterloader 77 for controlling or adapting the position of the gamma source for matching the pre-planned position. It will be appreciated that a suitable pre-planned position is established before implementing the treatment for effectuating the pre-determined treatment plan. The pre-planned source position is carried out by a suitable dose planning system based on the patient images.

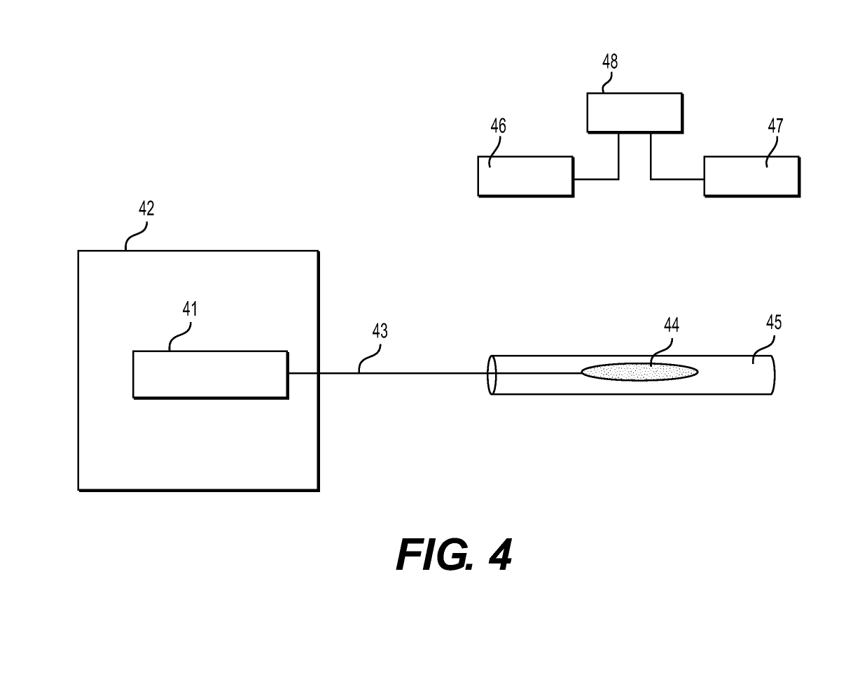

FIG. 4 presents in a schematic way an embodiment of a displaceable object according to an aspect of the present disclosure. In this embodiment, a suitable afterloading device 42 comprises the apparatus as is discussed with reference to the foregoing. The displaceable object 44 may be arranged to mimic the actual radiotherapeutic source used by the afterloader device 42 for effectuating treatment. The object 44 may mimic the actual radiotherapeutic source in terms of geometry and/or isotope. However, it may be preferable to use an object that has a lower activity than the isotope used for actual treatment. This may prevent the patient from receiving any unnecessary radiation and may allow personnel to safely be present in the room while performing the measurements.

In a different embodiment, the object may be configured to emit ultrasound or non-ionizing electromagnetic radiation. Again, the object used may mimic, represent, or simulate the geometry and size of the radioactive source used during treatment. By using an appropriately sized object, the object may also be used to check whether the conduits may receive the radioactive source properly.

When the object is transported inside a suitable conduit 45, for example, using a suitable cable 43 of the afterloader device, radiation emitted by the object is detected by suitable detectors 46, 47. Data from the detectors is supplied to a processor 48 for further processing. The processor 48 may be arranged to control the afterloading device for controlling the displacement of the object 44 inside the conduit 45. In this way, an automatic, real time feed-back between the apparatus may be used for determining the position of the conduit and the afterloader 42.

While specific embodiments have been described above, it will be appreciated that the invention may be practiced otherwise than as described. The descriptions above are intended to be illustrative, not limiting. Thus, it will be apparent to one skilled in the art that modifications may be made to the invention as described in the foregoing without departing from the scope of the claims set out below.

We claim:

1. A method for reconstructing a spatial position of a conduit arranged to accommodate a radiotherapeutic radioactive source, the method comprising:
displacing an object emitting radiation inside the conduit;
detecting said radiation;
generating data upon detecting the said radiation; and
reconstructing the spatial position of the conduit based on the said data.

2. The method according to claim 1, wherein the said radiation is electromagnetic radiation or ultrasonic radiation.

3. The method according to claim 1, wherein the electromagnetic radiation is gamma radiation.

4. The method according to claim 1, wherein the conduit is an interstitial needle, an applicator, or a catheter, the method further comprising the steps of:
accessing a pre-plan calculated for the conduit;
calculating a net dose distribution based on the pre-plan and the reconstructed spatial position of the conduit.

5. The method according to claim 1, wherein a plurality of conduits is used having individual spatial positions.

6. The method according to claim 1, wherein the object emitting radiation is arranged to mimic the radiotherapeutic radioactive source to be administered in the conduit during treatment.

7. The method according to claim 6, wherein the object emitting radiation is a radioactive source and has a substantially lower dose rate than the said radiotherapeutic radioactive source.

8. The method according to claim 6, wherein the object emitting radiation has substantially the same dimension as the radioactive source to be accommodated in the conduit.

9. The method according to claim 1, further comprising the step of creating a three-dimensional map of the conduit to verify at least the pre-determined dwell positions of a radiotherapeutic source inside the conduit, said dwell positions being provided by a treatment plan.

10. The method according to claim 9, wherein said map is obtained using a measurement cable of an afterloading device.

11. The method according to claim 9, wherein a plurality of conduits is used, each conduit having a pre-determined three-dimensional position according to the treatment plan, the method further comprising the step of verifying whether the actual three-dimensional map of each conduit matches the pre-determined three-dimensional position,

12. The method according to claim 1, further comprising the steps of:
displacing the radiotherapeutic radioactive source inside the conduit,
detecting radiation emitted by the radioactive source;
reconstructing the total dose delivered by the radiotherapeutic radioactive source.

13. An apparatus for enabling quality assurance of a brachytherapy treatment wherein a conduit adapted to accommodate a radioactive source is provided, the apparatus comprising:
a displaceable object adapted to be displaced inside the conduit, said object emitting radiation,
a detector capable of detecting radiation from the object;
a processor for reconstructing the spatial position of the conduit based on the output of the said detector.

14. The apparatus according to claim 13, wherein the object is a mimic of a radiotherapeutic radioactive source.

15. The apparatus according to claim 14, wherein said object has a substantially lower dose rate than the radiotherapeutic radioactive source.

16. The apparatus according to claim 15, wherein the processor is adapted for reconstructing the total dose delivered by the radiotherapeutic radioactive source accommodated and displaced in the conduit.

17. An afterloader device for effectuating a brachytherapy treatment using a radioactive source, comprising the apparatus according to claim 13.
